(19) United States
(12) Patent Application Publication
(10) Pub. No.: US 2025/0054639 A1
(43) Pub. Date: Feb. 13, 2025

(54) COMPUTER-IMPLEMENTED METHOD FOR THE SIMULATION OF MYOCARDIAL BLOOD FLOW UNDER STRESS CONDITIONS

(71) Applicant: CENTRO CARDIOLOGICO MONZINO S.p.A., Milano (MI) (IT)

(72) Inventors: Simone DI GREGORIO, Milano (MI) (IT); Gianluca PONTONE, Milano (MI) (IT); Alfio QUARTERONI, Milano (MI) (IT); Christian VERGARA, Milano (MI) (IT)

(73) Assignee: CENTRO CARDIOLOGICO MONZINO S.p.A., Milano (MI) (IT)

(21) Appl. No.: 18/720,084

(22) PCT Filed: Dec. 12, 2022
(86) PCT No.: PCT/IB2022/062070

§ 371 Date: Jun. 14, 2024

(30) Foreign Application Priority Data
Dec. 15, 2021 (IT) 102021000031475

(51) Int. Cl. G16H 50/50

(52) U.S. Cl. CPC G16H 50/50

(57) ABSTRACT

A computer-implemented method for the simulation of myocardial blood flow under stress conditions comprises a step of generating a simulated multi-physics model of a myocardial perfusion, wherein the step of generating further comprises: a step of generating a simulated model of the epicardial vessels based on a three-dimensional fluid-dynamics description; a step of generating a simulated model of the intramural vessels based on a multi-compartment porous medium; a step of coupling the simulated model of the epicardial vessels and the simulated model of the intramural vessels; a step of automatic calibration of physical parameters of the simulated multi-physics model of a myocardial perfusion under stress conditions, wherein the calibrated physical parameters are permeability tensors, conductances between the epicardial coronary arteries and the intramural vessels, and inter-compartment conductances between the compartments.

### TECHNICAL FIELD OF THE INVENTION

The present invention relates to a computer-implemented method for the simulation of myocardial blood flow under stress conditions.

### STATE OF THE ART

The myocardial perfusion, also known as myocardial blood flow (MBF), is the delivery of blood to the heart muscle, named myocardium, supplied by the coronary circulation.

The quantification of MBF and the functional assessment of coronary artery disease (CAD) could be achieved through stress myocardial computed tomography perfusion (stress-CTP).

This technique requires an additional scan after the coronary computed tomography angiography at rest (cCTA) and an intravenous stressor administration, leading to an increase of radiation exposure for the patient and potential stressor's related side effects.

Computational methods could reveal an effective tool as a concrete support for clinicians, allowing a completely noninvasive diagnostic technique for myocardial perfusion quantification and for coronary stenosis detection.

To this purpose, in document “A computational model applied to myocardial perfusion in the human heart: From large coronaries to microvasculature.” (Journal of Computational Physics, 424:109836, 2021), a multi-physics mathematical and numerical model of myocardial perfusion was proposed to quantify MBF avoiding the stress protocol and the related potential side effects and reducing the radiation exposure.

However, the mathematical and numerical model per se is not sufficient to quantify MBF in stress conditions, indeed it requires a suitable set of patient-specific parameters, which can allow the numerical simulations to compute MBF in stress conditions.

### SUMMARY OF THE INVENTION

Therefore, the main aim of the present invention is to provide a computer-implemented method for the simulation of myocardial blood flow under stress conditions that allows to compute MBF in stress conditions in an effective and accurate way for the specific patient.

The above-mentioned objects are achieved by the present computer-implemented method for the simulation of myocardial blood flow under stress conditions according to the features of claim 1.

### DESCRIPTION OF THE FIGURES

Other characteristics and advantages of the present invention will become better evident from the description of a preferred, but not exclusive embodiments of a computer-implemented method for the simulation of myocardial blood flow under stress conditions, illustrated by way of an indicative but non-limiting example in the accompanying Figures, in which:

FIG. 1 is a block diagram of the computer-implemented method according to the invention;

FIG. 2 schematically shows vasodilation under stress conditions simulated for a physical parameters adjustment step of the computer-implemented method according to the invention.

### DETAILED DESCRIPTION OF PREFERRED EMBODIMENTS

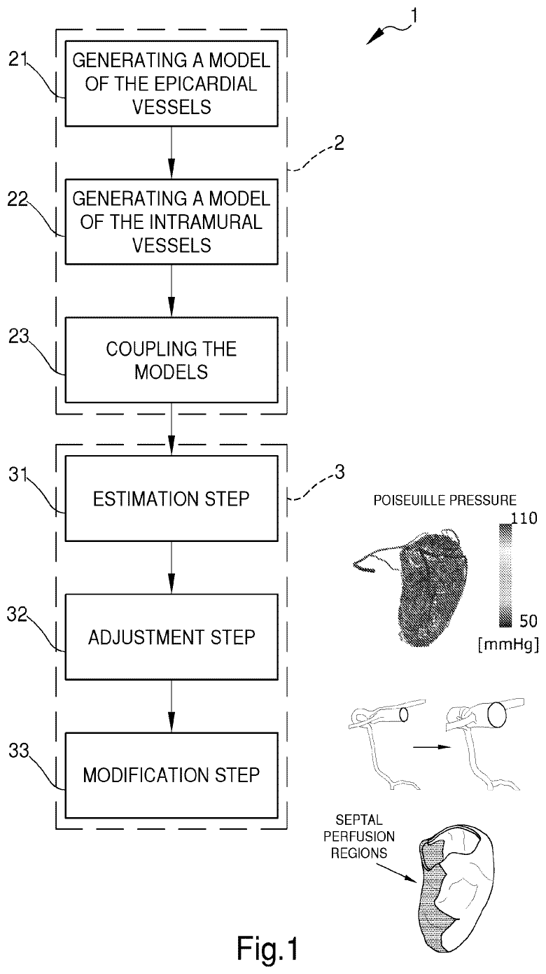

With particular reference to FIG. 1, globally indicated with reference 1 is a computer-implemented method for the simulation of myocardial blood flow under stress conditions.

The computer-implemented method 1 according to the invention comprises a first step 2 of generating a simulated multi-physics model of a myocardial perfusion.

Particularly, in the coronary artery tree a clear scale separation can be observed between the main vessels laying on the epicardium, the epicardial vessels, and the smaller vessels penetrating into the tissue, the intramural vessels (The multi-scale modelling of coronary blood flow. Annals of Biomedical Engineering, 40(11):2399-2413, 2012).

Therefore, because of such scale separation, the step 2 of generating the simulated multi-physics model of a myocardial perfusion comprises:
a step 21 of generating a simulated model of the epicardial vessels by means of a three-dimensional fluid-dynamics description;
a step 22 of generating a simulated model of the intramural vessels by means of a multi-compartment porous medium.

According to a preferred embodiment, the step of generating a simulated model of the epicardial vessels using a three-dimensional fluid-dynamics description is implemented using incompressible Navier-Stokes equations.

Furthermore, according to the preferred embodiment, the step of generating a simulated model of the intramural vessels by means of a multi-compartment porous medium is implemented using Darcy's law (Multi-scale parameterisation of a myocardial perfusion model using whole-organ arterial networks. Annals of Biomedical Engineering, 42(4):797-811, 2014).

Furthermore, the step 2 of generating the multi-physics simulated model of a myocardial perfusion comprises a step 23 of coupling the simulated model of the epicardial vessels and the simulated model of the intramural vessels using interface conditions based on the continuity of mass and momentum in every perfusion regions, wherein each perfusion region is a specific myocardial territory perfused by a distinct epicardial vessel (Visualisation of intramural coronary vasculature by an imaging cryomicrotome suggests compartmentalization of myocardial perfusion areas. Medical and Biological Engineering and Computing, 43(4):431-435, 2005).

Particularly, in case of three compartments, the step 2 of generating a multi-physics simulated model of a myocardial perfusion can be implemented by executing the following expressions:

$$\begin{matrix}ρ ( \frac{\partial {u}_{C}}{\partial t} + ( {u}_{C} \cdot ∇ ) {u}_{C} ) - μ ∇ \cdot ( {∇}_{{u}_{C}} + {( {∇}_{u} C )}^{T} ) + {∇}_{{p}_{C}} = 0 & in {Ω}_{C} , \\ ∇ \cdot {u}_{C} = 0 & in {Ω}_{C} , \\ {p}_{C} - μ ( {∇}_{{u}_{C}} + {( {∇}_{{u}_{C}} )}^{T} ) n \cdot n - \frac{1}{{a}^{j}} {\int }_{{Γ}^{j}} {u}_{C} \cdot nd γ = \frac{1}{\vert {Ω}_{M}^{j} \vert} {\int }_{{Ω}_{M}^{j}} {p}_{M , 1} dx & on {Γ}^{j} , \\ \begin{matrix}μ ( {∇}_{{u}_{C}} + {( {∇}_{{u}_{C}} )}^{T} ) n \cdot {τ}_{i} = 0 & i = 1 , 2\end{matrix} & on {Γ}^{j} . \\ {u}_{M , 1} + {K}_{1} ∇ {p}_{M , 1} = 0 & in {Ω}_{M} , \\ ∇ \cdot {u}_{M , 1} = {\sum }_{j = 1}^{J} \frac{{χ}_{{Ω}_{M}^{j}}}{\vert {Ω}_{M}^{j} \vert} {\int }_{{Γ}^{j}} {u}_{C} \cdot nd γ - {β}_{1 , 2} ( {p}_{M , 1} - {p}_{M , 2} ) & in {Ω}_{M} , \\ {u}_{M , 2} + {K}_{2} ∇ {p}_{M , 2} = 0 & in {Ω}_{M} , \\ ∇ \cdot {u}_{M , 2} = - {β}_{2 , 1} ( {p}_{M , 2} - {p}_{M , 1} ) - {β}_{2 , 3} ( {p}_{M , 2} - {p}_{M , 3} ) & in {Ω}_{M} , \\ {u}_{M , 3} + {K}_{3} ∇ {p}_{M , 3} = 0 & in {Ω}_{M} , \\ ∇ \cdot {u}_{M , 3} = - γ ( {p}_{M , 3} - {p}_{veins} ) - {β}_{3 , 2} ( {p}_{M , 3} - {p}_{M , 2} ) & in {Ω}_{M} ,\end{matrix}$$

wherein:
Ω_{C} is the domain of the epicardial coronary arteries;
Ω_{M} is the domain of the intramural vessels;
Ω^{J}_{M}, j=1, . . . , J, are the domains of the perfusion regions; u_{C} and p_{C} are the blood velocity and pressure, respectively, in the epicardial coronary arteries;
ρ is the blood density;
μ is the blood viscosity;
n is the unit outer normal vector;
αⁱ are the conductances between the epicardial coronary arteries and the intramural vessels.

Furthermore, for i=1 . . . 3 in the i-th compartment:
u_{M,i} and p_{M,i} are the Darcy velocity and the pore pressure, respectively;
Kᵢ is the permeability tensor;
βᵢ, k≥0, i, k=1 . . . N, represent the inter-compartment pressure-coupling coefficients between the compartments i and k;
ρᵥₑᵢₙₛ is the venous pressure;
γ is a suitable drain coefficient.

Finally, the notation χ_{A} stands for the characteristic function of the domain A.

Particularly, to enforce mass conservation among compartments, we have that βi, k=βk, i, ∀i, k=1, . . . , N.

Moreover, β_{i, k}≠0 whenever k=i±1 for 2≤i≤N−1, k=2 for i=1, k=N−1 for i=N, since the compartment i exchanges mass only with adjacent compartments. Particularly, only the first compartment is involved in the coupling condition and the average of pressure on the whole compartment Ωj is considered due to the homogenized nature of the Darcy equation.

Particularly, it is pointed out that the step 2 of generating the multi-physics simulated model can be implemented as disclosed in the document “A computational model applied to myocardial perfusion in the human heart: From large coronaries to microvasculature (Journal of Computational Physics, 424:109836, 2021).

To reproduce clinical data of a myocardial blood flow (MBF) maps, the simulation implemented by the computer-implemented method 1 according to the invention require a proper set of specific physical parameters of the simulated multi-physics model of a myocardial perfusion.

Advantageously, the computer-implemented method 1 according to the invention comprises a step 3 of automatic calibration of physical parameters of the multi-physics simulated model of a myocardial perfusion under stress conditions.

Particularly, said calibrated physical parameters are:
the permeability tensors Kᵢ, i=1, 2, 3;
the conductances αⁱ, j 1, . . . , J, between the epicardial coronary arteries and
the intramural vessels; and
the inter-compartment conductances β_{i,k}, i, k=1, 2, 3, between the compartments i and k.

Suitable values of such parameters are estimated for each patient and assumed to vary among the different perfusion regions.

As schematically showed in FIG. 1, the step 3 of automatic calibration of the physical parameters under stress conditions comprises the following steps:
an estimation step 31 of the physical parameters in rest conditions, by exploiting the intramural vessels geometrical and fluid dynamics properties in rest conditions;
an adjustment step 32 of the physical parameters accounting vasodilation under stress conditions;
a modification step 33 of the physical parameters at the septum, particularly by increasing of physical parameters in the septum.

According to the estimation step 31, the intramural vascular network is exploited to estimate first possible values for the physical parameters in rest conditions.

As for the permeability tensors Kᵢ, i=1, 2, 3, they are initialized based on geometric issues, in particular for each compartment and perfusion region they were given by the ratio between the volume of the intramural vessels in such region and the total region volume.

The first step consists in the separation of the intramural vascular network into two groups of vessels using a specific metric. This is motivated by the fact that it is possible to relate the largest vessels to the first compartment, whereas the smallest ones to the second compartment.

Particularly, it is pointed out that the surrogate intramural vascular network generated according to the document “A computational model applied to myocardial perfusion in the human heart: From large coronaries to microvasculature” (Journal of Computational Physics, 424:109836, 2021) does not include the microvasculature, which is the part of the network modelled in the third Darcy compartment.

To perform such operation, the estimation step 31 comprises a definition step of a hierarchic parameter ζ∈[0, 1] for each node yᵢ of the intramural vascular network.

Particularly, the definition step comprises calculating the hierarchic parameter ζ(yᵢ) as the ratio between the sum of the lengths of the vessels which are located distally to yᵢ and the sum of the lengths of all the vessels of the network.

In this way ζ will be 1 for the most proximal nodes and 0 for the most distal terminal nodes.

Then, given for each perfusion region Ω^{j}_{M} a value Z^{j}∈(0, 1), the estimation step 31 comprises a sorting step for sorting a vessel of the network to belong to the first or second group of vessels if the average of the values of ζ in the nodes of the vascular network at hand is in the range [0, Z^{j}] or in the range [Z^{j}, 1]. The values Z^{j} are chosen in order to have about the same number of vessels in the two groups.

Particularly, the estimation step 31 comprises calculating the global constant permeability tensor Kᵢ as:

$${K}_{i} ( x ) = {\sum }_{j = 1}^{J} {K}_{i}^{j} {χ}_{{Ω}_{M}^{j}} ( x )$$

where K^{j}ᵢ is the permeability tensor of the i-th compartment in the perfusion region Ω^{j}_{M}.

Particularly, the permeability tensor K^{j}ᵢ is assumed isotropic and is defined as:

$${K}_{i}^{j} = {ϕ}_{i}^{j} l$$

where I is the identity tensor with unit of cm²Pa⁻¹ s⁻¹ and Φ^{j}ᵢ is the constant porosity.

Particularly, the constant porosity Φ^{j}ᵢ is defined as follows:

$${ϕ}_{i}^{j} = \frac{{\sum }_{n = 1}^{{M}_{i}^{j}} {V}_{i , n}^{j}}{{V}_{{Ω}_{M}^{j}}} ,$$

where Vⱼ_{_{Ω}}_{^{M}} is the volume of Ω^{j}_{M}, M^{j}ᵢ is the number of the vessels in Ω^{j}_{M}, and V_{i,n}^{j} is the volume of the n-th vessel in the i-th compartment of Ω^{j}_{M}.

To compute the conductances β_{i, k} and α^{j}, other information about the pressure and flow distributions in the vascular network are required. Particularly, in order to find an approximation of the conductances β_{i, k} and α^{j}, the solution of a Poiseuille flow problem along vascular network is considered, given by the union between the epicardial coronaries and the intramural network.

To reduce the computational effort, the epicardial coronaries are modeled as 1D tubular structures (notice that this is done only for the parameter estimation, whereas elsewhere in the model the coronaries are 3D).

As for the boundary conditions, an inlet pressure of 109 mmHg and outlet pressures depending on the radius of the terminal vessels are prescribed.

A constant multiplicative correction factor η is in case applied to all the vessels radii in order to obtain a physiological flow rate starting from this pressure gradient.

Referring to this Poiseuille solution, Q^{j}_{i,k}=Q^{j}_{k,i}, k=1, 2, 3, is the total flow rate exchanged in the perfusion region Ω^{j}_{M} at the interface between compartment i and compartment k.

Since there are not any vessels in the third compartment, Ω^{j}_{2,3} is set to be equal to the total flow rate at the outlet of the second compartment of Ω^{j}_{M}.

Moreover, p^{j}_{i,n}, i=1, 2, is the pressure in the n-th vessel of compartment i in the perfusion region Ω^{j}_{M} and p̅₃^{j}=39 mmHg a reference value for the microvasculature pressure for allj, computed as the average value between the pressure of the most downstream vessels (≈56 mmHg) and the value of pᵥₑᵢₙₛ=22.5 mmHg.

Particularly, the estimation step 31 comprises calculating the global piecewise constant inter-compartment conductance β_{i, k} as:

$${β}_{i , k} ( x ) = {\sum }_{j = 1}^{J} {β}_{i , k}^{j} {χ}_{{Ω}_{M}^{j}} ( x )$$

where β^{j}_{i, k} is a local coupling coefficient.

Particularly, the local coupling coefficient β^{j}_{i, k} inside the perfusion region Ω^{j}_{M} is defined as:

$${β}_{1 , 2}^{j} = \{ \begin{matrix}0 & if {\overline{p}}_{1}^{j} - {\overline{p}}_{2}^{j} = 0 , \\ \frac{{\overline{Q}}_{1 , 2}^{j}}{\vert {\overline{p}}_{1}^{j} - {\overline{p}}_{2}^{j} \vert} & otherwise ,\end{matrix}$$

$${β}_{2 , 3}^{j} = \{ \begin{matrix}0 & if {\overline{p}}_{2}^{j} - {\overline{p}}_{3}^{j} = 0 , \\ \frac{{\overline{Q}}_{2 , 3}^{j}}{\vert {\overline{p}}_{2}^{j} - {\overline{p}}_{3}^{j} \vert} & otherwise ,\end{matrix}$$

$${β}_{i , k}^{j} = 0 \qquad elsewhere ,$$

$$where$$

$${\overline{Q}}_{i , k}^{j} = \frac{{Q}_{i , k}^{j}}{{V}_{{Ω}_{M}^{j}}}$$

$$and$$

$${\overline{p}}_{i}^{j} = \frac{{\sum }_{n = 1}^{{M}_{i}^{j}} {p}_{i , n}^{j} {V}_{i , n}^{j}}{{\sum }_{n = 1}^{{M}_{i}^{j}} {V}_{i , n}^{j}} i = 1 , 2$$

In a similar way, the estimation step 31 comprises calculating the conductance coefficient α^{j} as:

$${α}^{j} = \frac{{Q}_{inlet}^{j}}{\vert {p}_{inlet}^{j} - {\overline{p}}_{1}^{j} \vert} , j = 1 , … , J$$

where Q^{j}ᵢₙₗₑₜ is the flow rate entering in the first compartment of Ω^{j}_{M} and p^{j}ᵢₙₗₑₜ is the pressure in the first node of the first compartment of Ω^{j}_{M}.

After computing the physical parameters under rest conditions, the stressor agent effect on the coronary arteries shall be accounted. To this purpose the epicardial coronary artery domain Ω_{C}, which was reconstructed from rest CT images, was post-processed to account for the vasodilation.

Particularly, the adjustment step 32 of the physical parameters comprises executing the following steps:
choosing a sample epicardial coronary artery, which is visible on axial scans acquired under rest and stress conditions;
measuring the value of the radius under rest conditions Rᵣₑₛₜ₋ₛₐₘₚₗₑ and the value of the radius under stress conditions Rₛₜᵣₑₛₛ₋ₛₐₘₚₗₑ in the sample epicardial coronary artery;
compute the vasodilation factor νₛₜᵣ as

$${υ}_{str} = \frac{{R}_{stress - sample}}{{R}_{rest - sample}}$$

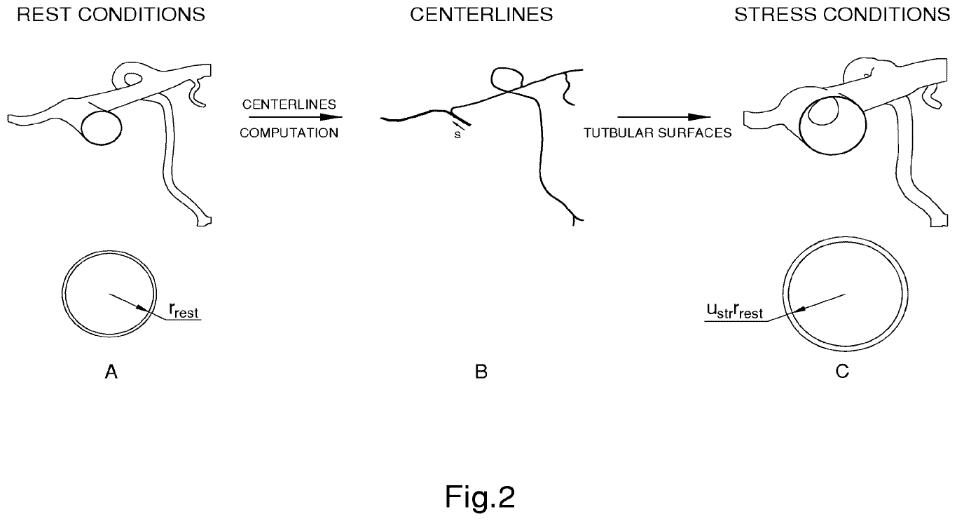

compute the centerlines of the epicardial coronary arteries reconstructed from rest computed tomography angiography (CTA) and compute the radius of the vessels in each point of the centerlines (FIG. 2 from A to B);
generate a new epicardial coronary arteries surface by extruding a tubular surface from the centerlines (FIG. 2 from B to C), whose radius in each tract is computed as

$${r}_{stress} ( s ) = {υ}_{str} {r}_{rest} ( s )$$

where s is the curvilinear abscissa along the centerlines.

Moreover, according to physiological evidences, the adenosine injection leads to an increase of the vascular resistance of about 10-fold. For this reason, in order to account for the vasodilation in the intramural vascular network, the physical resistive parameters of the multi-compartment Darcy model β_{1,2} and β_{2,3}, K₁ and K₂ are increased by 10-fold with respect to baseline parameters estimated for resting conditions at said estimation step.

A final adjustment is performed based on the observation that at the septum the values of β_{1,2} and β_{2,3} estimated at said adjustment step are lower than the other myocardial regions, leading to a systematic underestimation of MBF computed by the numerical simulations with respect to MBF estimated with stress-CTP.

To overcome this, the modification step 33 comprises multiplying the inter-compartment pressure-coupling coefficients β_{1,2} and β_{2,3} by a factor 5 in perfusion regions located at the ventricular septum.

The present invention is also related to an apparatus for coronary computed tomography angiography at rest (cCTA) configured for executing the steps of the computer-implemented method 1 as disclosed above.

Therefore, the apparatus according to the invention comprises all the hardware and software conventionally needed for the coronary computed tomography angiography at rest (cCTA) and a further elaboration unit configured for executing he computer-implemented method 1.

1) A computer-implemented method for simulation of myocardial blood flow under stress conditions, executed on an apparatus for coronary computed tomography angiography at rest, comprising a step of generating a simulated multi-physics model of a myocardial perfusion, wherein said step of generating said simulated multi-physics model of the myocardial perfusion further comprises:
a step of generating a simulated model of epicardial vessels by means of a three-dimensional fluid-dynamics description;
a step of generating a simulated model of intramural vessels by means of a multi-compartment porous medium;
a step of coupling the simulated model of the epicardial vessels and the simulated model of the intramural vessels;
a step of automatic calibration of physical parameters of the simulated multi-physics model of a myocardial perfusion under stress conditions, wherein said calibrated physical parameters are:
permeability tensors (Kᵢ, i=1, 2, 3);
conductances between epicardial coronary arteries and the intramural vessels (α^{j},j=1, . . . , J); and
inter-compartment conductances (β_{i,k}, i, k=1, 2, 3) between compartments (i, k);
wherein said step of automatic calibration comprises the following steps:
an estimation step of the physical parameters in rest conditions, by exploiting the intramural vessels geometrical and fluid dynamics properties in rest conditions;
an adjustment step of the physical parameters accounting vasodilation under stress conditions;
a modification step of the physical parameters at a septum, by increasing of physical parameters in the septum;
and wherein said adjustment step of the physical parameters comprises executing the following steps:
choosing a sample epicardial coronary artery, which is visible on axial scans acquired under rest and stress conditions;
measuring a value of a radius under rest conditions Rᵣₑₛₜ₋ₛₐₘₚₗₑ and a value of a radius under stress conditions Rₛₜᵣₑₛₛ₋ₛₐₘₚₗₑ in the sample epicardial coronary artery;
compute the vasodilation factor νₛₜᵣ as
$${v}_{str} = \frac{{R}_{stress - sample}}{{R}_{rest - sample}}$$
compute centerlines of the epicardial coronary arteries reconstructed from rest computed tomography angiography (CTA) and compute the radius of the vessels in each point of the centerlines;
generate a new epicardial coronary arteries surface by extruding a tubular surface from the centerlines, whose radius in each tract is computed as
$${r}_{stress} ( s ) = {v}_{str} {r}_{rest} ( s )$$
where s is a curvilinear abscissa along the centerlines.

2) The computer-implemented method according to claim 1, wherein in said estimation step comprises calculating the global constant permeability tensor Kᵢ as:
$${K}_{i} ( x ) = {\sum }_{j = 1}^{J} {K}_{i}^{j} {χ}_{{Ω}_{M}^{j}} ( x )$$
where K^{j}ᵢ is a permeability tensor of an i-th compartment in a perfusion region Ω^{j}_{M}.

3) The computer implemented method according to claim 2, wherein said permeability tensor K^{j}ᵢ is defined as:
$${K}_{i}^{j} = {ϕ}_{i}^{j} l$$
where I is an identity tensor with unit of cm² Pa⁻¹ s⁻¹ and Φ^{j}ᵢ is a constant porosity.

4) The computer-implemented method according to claim 3, wherein said constant porosity Φ^{j}ᵢ is defined as follows:
$${ϕ}_{i}^{j} = \frac{{\sum }_{n = 1}^{{M}_{i}^{j}} {V}_{i , n}^{j}}{{V}_{{Ω}_{M}^{j}}} ,$$
where Vⱼ_{_{Ω}}_{^{M}} is a volume of Ω^{j}_{M}, M^{j}ᵢ is a number of vessels in Ω^{j}_{M}, and V_{i,n}^{j} is a volume of a n-th vessel in an i-th compartment of Ω^{j}_{M}.

5) The computer-implemented method according to claim 4, wherein said estimation step comprises calculating a global piecewise constant inter-compartment conductances β_{i, k} as:
$${β}_{i , k} ( x ) = {\sum }_{j = 1}^{J} {β}_{i , k}^{j} {χ}_{{Ω}_{M}^{j}} ( x )$$
where β^{j}_{i, k} is a local coupling coefficient.

6) The computer-implemented method according to claim 5, wherein said local coupling coefficient β^{j}_{i,k} inside the perfusion region Ω^{j}_{M} is defined as:
$${β}_{1 , 2}^{j} = \{ \begin{matrix}0 & if {\overline{p}}_{1}^{j} - {\overline{p}}_{2}^{j} = 0 , \\ \frac{{\overline{Q}}_{1 , 2}^{j}}{\vert {\overline{p}}_{1}^{j} - {\overline{p}}_{2}^{j} \vert} & otherwise ,\end{matrix}$$
$${β}_{2 , 3}^{j} = \{ \begin{matrix}0 & if {\overline{p}}_{2}^{j} - {\overline{p}}_{3}^{j} = 0 , \\ \frac{{\overline{Q}}_{2 , 3}^{j}}{\vert {\overline{p}}_{2}^{j} - {\overline{p}}_{3}^{j} \vert} & otherwise ,\end{matrix}$$
$${β}_{i , k}^{j} = 0 \qquad elsewhere ,$$
$$where$$
$${\overline{Q}}_{i , k}^{j} = \frac{{Q}_{i , k}^{j}}{{V}_{{Ω}_{M}^{j}}}$$
$$and$$
$${\overline{p}}_{i}^{j} = \frac{{\sum }_{n = 1}^{{M}_{i}^{j}} {p}_{i , n}^{j} {V}_{i , n}^{j}}{{\sum }_{n = 1}^{{M}_{i}^{j}} {V}_{i , n}^{j}} i = 1 , 2$$

7) The computer-implemented method according to claim 6, wherein said estimation step comprises calculating the conductance coefficient α^{j} as:
$${α}^{j} = \frac{{Q}_{inlet}^{j}}{\vert {p}_{inlet}^{j} - {\overline{p}}_{1}^{j} \vert} , j = 1 , … , J$$
where Q^{j}ᵢₙₗₑₜ is a flow rate entering in a first compartment of Ω^{j}_{M} and p^{j}ᵢₙₗₑₜ is a pressure in a first node of the first compartment of Ω^{j}_{M}.

8) The computer-implemented method according to claim 7, wherein said modification step Comprises multiplying inter-compartment pressure-coupling coefficients (β_{1,2}, β_{2,3}) by a predefined factor in septal perfusion regions.

9) The apparatus for coronary computed tomography angiography at rest (cCTA) configured for executing the steps of the computer-implemented method for the simulation of myocardial blood flow under stress conditions according to claim 1.

10) The computer-implemented method according to claim 2, wherein said estimation step comprises calculating a global piecewise constant inter-compartment conductances β_{i, k} as:
$${β}_{i , k} ( x ) = {\sum }_{j = 1}^{J} {β}_{i , k}^{j} {χ}_{{Ω}_{M}^{j}} ( x )$$
where β^{j}_{i, k} a local coupling coefficient.

11) The computer-implemented method according to claim 1, wherein said modification step comprises multiplying inter-compartment pressure-coupling coefficients (β_{1,2}, β_{2,3}) by a predefined factor in septal perfusion regions.

12) A non-transitory computer readable medium having instructions stored thereon, such that when the instructions are read and executed by one or more processors, said one or more processors is configured to perform the computer-implemented method according to claim 1.
